(19) United States
(12) Patent Application Publication
(10) Pub. No.: US 2009/0131813 A1
(43) Pub. Date: May 21, 2009

(54) Method and apparatus for augmenting nerve stimulation and response detection

(76) Inventors: Marc P. Cryan, Maynard, MA (US); Charles Fendrock, Sudbury, MA (US); Paul Brefka, Southborough, MA (US)

Correspondence Address: Mark J. Pandiscio;Pandiscio & Pandiscio, P.C., 470 Totten Pond Road, Waltham, MA, 02451-1914, US

(21) Appl. No.: 12/215,642

(22) Filed: Jun. 27, 2008

Related U.S. Application Data
(60) Provisional application No. 60/937,915, filed on Jun. 29, 2007.

(51) Int. Cl. A61B 5/05

(52) U.S. Cl. 600/554

(57) ABSTRACT

Apparatus for use in nerve conduction testing where a stimulating electrode is used to electrically stimulate a nerve and a detection electrode is used to detect an evoked neurological response, the apparatus comprising:
a shaped protrusion for positioning against an electrode; and
a compression mechanism for applying a force to the shaped protrusion so as to cause the shaped protrusion to press the electrode against the underlying tissue and compress the same, so as to move the electrode closer to the nerve and thereby augment nerve stimulation and response detection.

### REFERENCE TO PENDING PRIOR PATENT APPLICATION

This patent application claims benefit of pending prior U.S. Provisional Patent Application Ser. No. 60/937,915, filed Jun. 29, 2007 by Marc P. Cryan et al. for NERVE STIMULATION AUGMENTATION APPARATUS AND METHOD (Attorney's Docket No. NEURO-26 PROV), which patent application is hereby incorporated herein by reference.

### FIELD OF THE INVENTION

This invention relates to apparatus and methods for the assessment of neuromuscular function. More specifically, this invention relates to apparatus and methods for improving the effectiveness and efficiency of the electrical stimulation and response detection for peripheral nerves in patients, especially those patients with large limbs, in order to diagnose peripheral nerve and muscle pathologies based on assessments of neuromuscular or neurosensory function.

### BACKGROUND OF THE INVENTION

There are many clinical and non-clinical situations that call for the rapid, reliable and low-cost assessment of neuromuscular and neurosensory function. The most common causes of neuromuscular and neurosensory disruption are related to pathologies of the peripheral nerves and muscles. Carpal tunnel syndrome (CTS) is one of the most common forms of neuromuscular disease. This disease is thought to arise from compression of the median nerve as it traverses the wrist. CTS causes discomfort or loss of sensation in the hand and, in severe cases, a nearly complete inability to use one's hands. Highly repetitive wrist movements are thought to be one cause of CTS. Other widespread medical conditions such as, for example, diabetes, rheumatoid arthritis and cancer, are associated with neuromuscular disease.

Neuromuscular disorders such as, for example, Carpal Tunnel Syndrome (CTS), are very common and well known to the general public. Neuromuscular function is generally assessed by electrically stimulating a nerve and then measuring the response of a muscle innervated by that nerve. The muscle response is detected by measuring the myoelectric potential generated by the muscle in response to the stimulus. Similarly, a sensory nerve is generally assessed by electrically stimulating the sensory nerve and then measuring the response of the nerve, preferably directly over the nerve at a known distance from the stimulation site. One indication of the physiological state of the nerve is provided by the delay between the application of a stimulus and the detection of a muscular or sensory response. If the nerve is damaged, conduction of the signal via the nerve to the muscle and, hence, detection of the muscle's response, or conduction of the signal via the nerve to the sensory detection site, will be slower than in a healthy nerve. An abnormally high delay between the stimulus application and the detection of the muscle or sensory nerve response indicates, therefore, impaired neuromuscular function.

Reliable automated devices have been introduced into the marketplace to assess neuromuscular function in primary care physician and small clinic settings. These automated devices are intended to be used by personnel without specialized training. The apparatus and method described by Gozani in U.S. Pat. No. 5,976,094 is one example of a device and method that are currently successfully used to make neuromuscular assessments of peripheral nerves many thousands of times every year. The method and device described by Gozani utilizes a set of stimulation electrodes that are in a fixed geometric relationship and that are proximal to a set of detection electrodes. The detection electrodes detect the myoelectrical potential that is evoked by the electrical stimulus.

The electrical stimulation current must travel through the tissue mass between the electrodes on the surface of the skin and the nerve within the limb. The evoked myoelectrical potential is a signal that is conducted through the volume of body tissue that lies between the stimulated nerve and the detection electrodes on the surface of the skin and is useful in helping to diagnose neurological disorders.

It is a desirable goal for the medical practitioner to evoke the maximum nerve response for the smallest stimulation current and to minimize patient discomfort caused by an excessively large stimulus, especially in the limbs of patients that are large due to obesity or muscle structure. Because the stimulation current travels across a several centimeter distance of body tissue, the current density of the stimulation current is frequently too low to cause a sufficient evoked response from the nerve to be useful for diagnosis. In many cases a response waveform with sufficient amplitude and suitable waveform cannot be obtained from the maximum stimulus that the test instrument can produce because the distance between the stimulation electrodes on the skin and the nerve within the limb, and the volume of the limb, is too large. An evoked waveform with greater amplitude and sharper definition would provide better information to make a more accurate diagnosis of the patient's condition.

In general clinical practice, in order to increase the amplitude of a signal evoked by a stimulus, a trained clinician can increase the stimulation current, however, there are practical limits to the maximum amplitude which can be generated by typical nerve testing equipment, and by the amount of discomfort that can be tolerated by the patient being tested.

An alternative approach is to improve the efficiency of a particular stimulation current through the use of finger pressure on the stimulation electrodes so as to push the intervening fat or muscle tissue out of the way and thereby move the stimulation electrodes closer to the nerve. However, this process is technique-dependent and must be performed by a skilled clinician trained in neurophysiology.

It is, therefore, an object of the present invention to provide a novel apparatus and method to increase the amplitude and waveform quality of the evoked neurological response for a given stimulus amplitude, especially in patients with large limbs, that can be quickly and effectively used by a minimally-trained medical practitioner.

### SUMMARY OF THE INVENTION

In accordance with the present invention, a novel apparatus and method are provided to improve the efficiency of stimulation and response detection of nerves in patients for a better assessment of neuromuscular function without the involvement of highly trained personnel.

With the apparatus and method of the present invention, once the medical practitioner has placed the stimulation and detection electrodes on the patient, the apparatus is then applied over the stimulation electrodes on the patient. The apparatus is tightened and automatically achieves a pressure that is in the desirable range so as to improve the efficiency of the stimulus and, therefore, the evoked response of the nerve being tested.

In a preferred embodiment, the invention consists of the provision and use of (i) a strap to gird the limb, (ii) a shaped protrusion that is placed over the stimulus electrodes and that presses against the flesh above the nerve and displaces the fat and muscle below it once the girding strap is tightened, thereby moving the stimulus electrodes closer to the nerve below, and (iii) a mechanism to indicate to the medical practitioner that the strap has been tightened with sufficient force that the resultant force on the protrusion, and hence the resulting pressure on the flesh, will be in an effective range.

The apparatus of the invention may be further embodied in a spring-loaded clamp that grabs and squeezes the limb, and that has a shaped protrusion that is placed over the stimulus electrodes and that presses against the flesh above the nerve and displaces the fat and muscle below the shaped protrusion once the clamp is deployed by the medical practitioner, thereby moving the stimulus electrodes closer to the nerve below.

In one preferred form of the invention, there is provided apparatus for use in nerve conduction testing where a stimulating electrode is used to electrically stimulate a nerve and a detection electrode is used to detect an evoked neurological response, the apparatus comprising:

a shaped protrusion for positioning against an electrode; and

a compression mechanism for applying a force to the shaped protrusion so as to cause the shaped protrusion to press the electrode against the underlying tissue and compress the same, so as to move the electrode closer to the nerve and thereby augment nerve stimulation and response detection.

In another form of the present invention, there is provided a system for testing a nerve, the system comprising:

a stimulating electrode for electrically stimulating a nerve;

a detection electrode for detecting an evoked neurological response;

a shaped protrusion for positioning against an electrode; and

a compression mechanism for applying a force to the shaped protrusion so as to cause the shaped protrusion to press the electrode against the underlying tissue and compress the same, so as to move the electrode closer to the nerve and thereby augment nerve stimulation and response detection.

In another form of the present invention, there is provided a method for augmenting nerve conduction testing where a stimulating electrode is used to electrically stimulate a nerve and a detection electrode is used to detect an evoked neurological response, the method comprising:

positioning a shaped protrusion against an electrode; and

positioning a compression mechanism against the shaped protrusion; and

activating the compression mechanism so as to apply a compressive force to the shaped protrusion, whereby to cause the shaped protrusion to press the electrode against the underlying tissue and compress the same, thereby moving the electrode closer to the nerve and augmenting nerve stimulation and response detection.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts and further wherein:

FIGS. 1 and 2 illustrate the location of a nerve within a limb of a patient, and the traditional positioning of the stimulation and detection electrodes on the surface of the limb, and illustrate the current density delivered to the nerve during stimulation;

FIGS. 3-5 illustrate a preferred embodiment of the present invention in use, and show the stimulation and detection electrodes compressed against the surface of the limb so as to lie closer to the nerve within the limb, and show the increased current density delivered to the nerve during stimulation;

FIGS. 6-10 provide additional construction details with respect to a preferred embodiment of the present invention;

FIG. 11 illustrates the amplitude (in millivolts) of a typical Compound Muscle Action Potential (CMAP) with and without the use of the present invention, for a given stimulation level;

FIG. 12 shows the improvement obtained in CMAP amplitude with use of the present invention, plotting stimulus current against compression force in order to obtain a supramaximal CMAP response;

FIG. 13 shows the reduction in stimulation intensity, by body mass index and applied force; and

FIG. 14 illustrates an alternative embodiment of the present invention.

### DETAILED DESCRIPTION OF THE INVENTION

As will be appreciated by those skilled in the art of performing neurological testing, it is generally desirable to employ a method and apparatus to increase the amplitude and waveform quality of the evoked neurological response for a given stimulus amplitude, where the method and apparatus can be quickly and effectively utilized by minimally-trained medical personnel. This is particularly true when performing nerve conduction testing on patients with large limbs (e.g., obese patients).

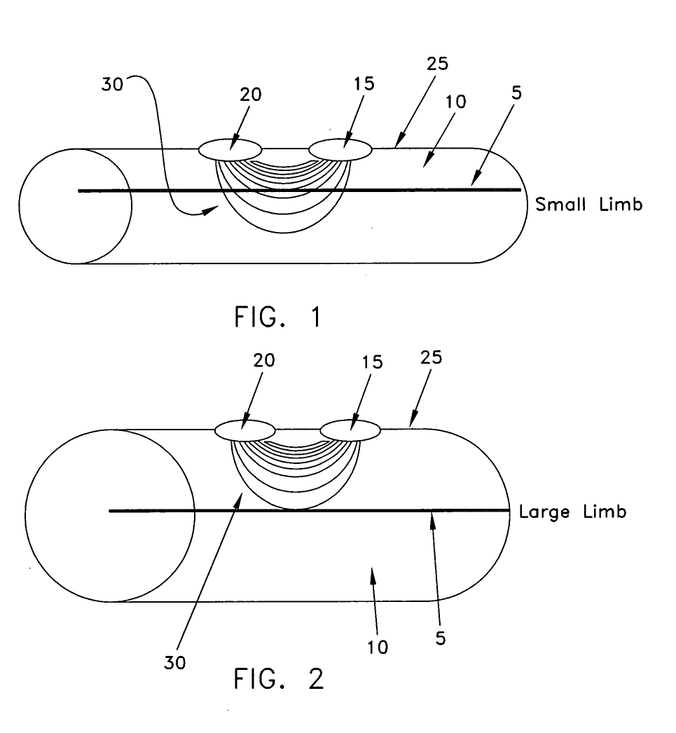

FIGS. 1 and 2 illustrate the location of a nerve 5 within a limb 10, and the traditional positioning of the stimulation and detection electrodes 15, 20, respectively, on the surface 25 of the limb, and illustrate the current density 30 delivered to the nerve during stimulation. As is well known in the art, it is common for the stimulation and the detection electrodes 15, 20 to be mounted on a single biosensor substrate, in order to facilitate quick and easy mounting of the stimulation and detection electrodes 15, 20 on the surface of the patient's skin. Furthermore, it is common for more than one stimulation electrode 15 and/or more than one detection electrode 20 to be provided on a given biosensor substrate, with one or more of the stimulation electrodes 15 and/or one or more of the detection electrodes 20 being selected for a given nerve conduction test. To this end, for the purposes of the present document, the term “electrode” is intended to include “electrodes” where the context so admits, and the term “electrodes” is intended to include “electrode” where the context so admits. As can be seen in FIGS. 1 and 2, the smaller the limb, the higher the current density delivered to the nerve and, therefore, the smaller the stimulus needed to achieve a maximum nerve response. With a larger limb, the opposite is true, with the required stimulus current increasing with increasing limb size.

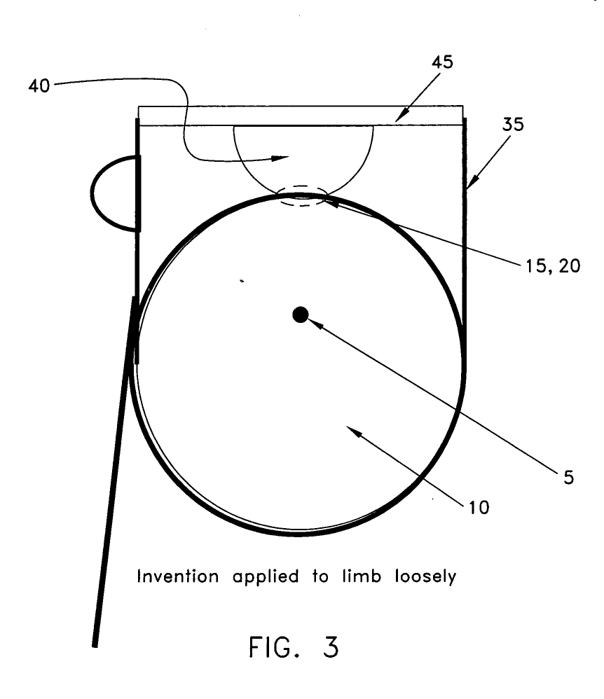

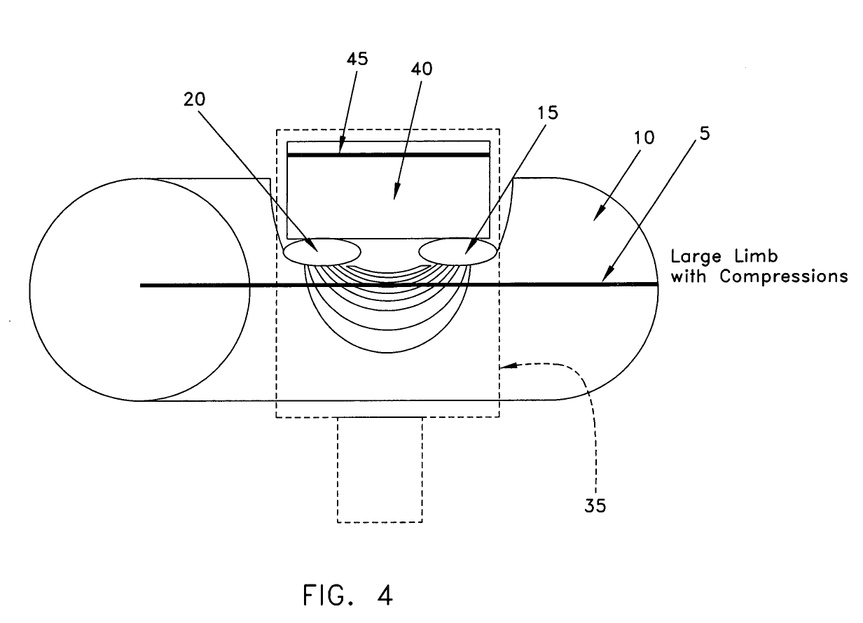

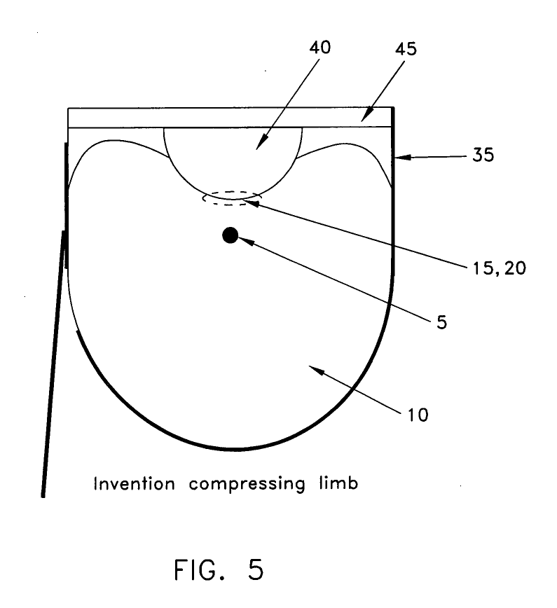

Looking next at FIGS. 3-5, the present invention comprises, in one preferred form of the invention, a strap 35 to gird the limb of the patient, a shaped protrusion 40 that is placed over the stimulus electrodes and presses the stimulus electrodes against the flesh of the patient above the nerve so as to displace the fat and muscle tissue disposed below the stimulus electrodes. Once girding strap 35 is tightened, thereby moving the shaped protrusion inwardly so as to force the stimulus electrodes closer to the nerve below, a mechanism (e.g., a mechanical stop) indicates to the medical practitioner that the strap has been tightened to a sufficient degree so that the resultant pressure delivered to the stimulus electrodes by shaped protrusion 40 will be sufficient to properly augment nerve stimulation and response detection. In one preferred form of the invention, the shaped protrusion can be backed by a rigid portion 45 which spans the width of the patient's limb and serves to concentrate the force generated by the girding strap over the shaped protrusion.

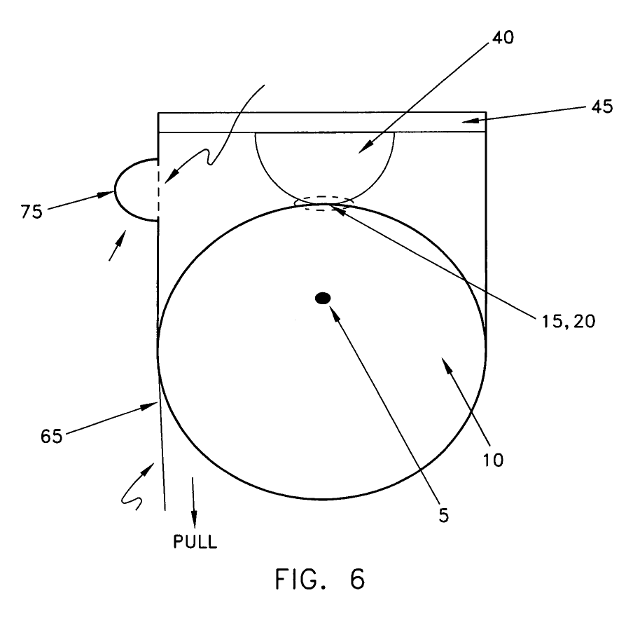

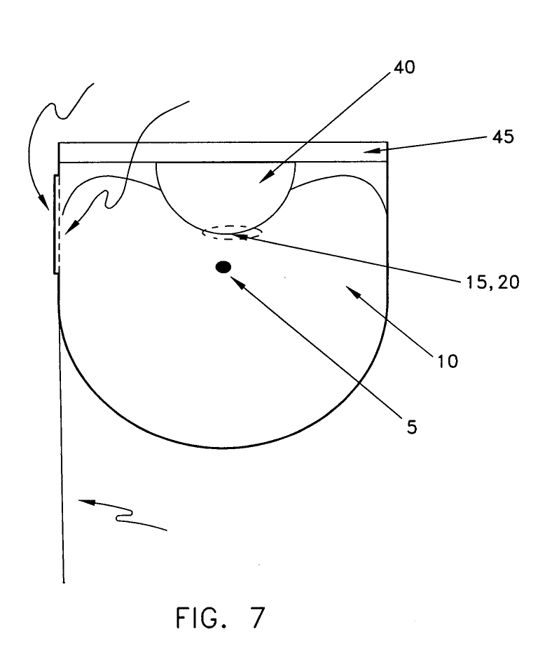

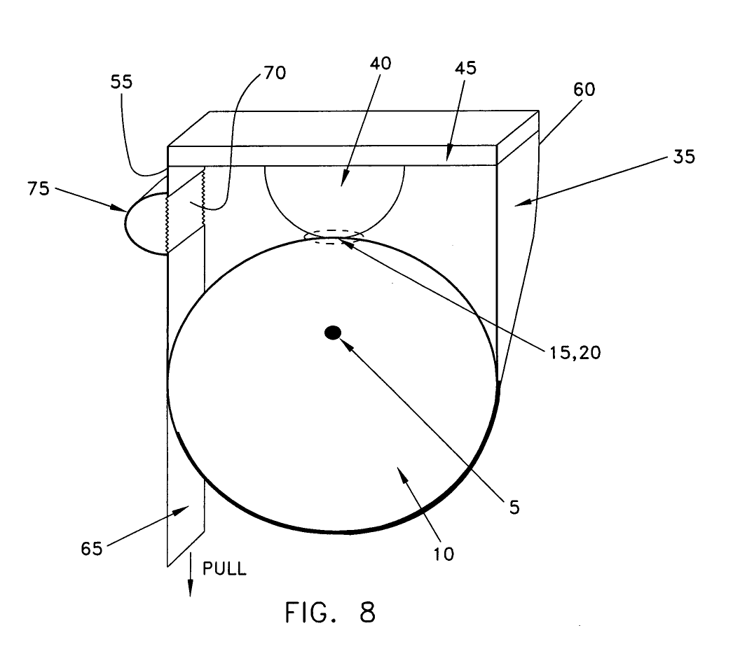

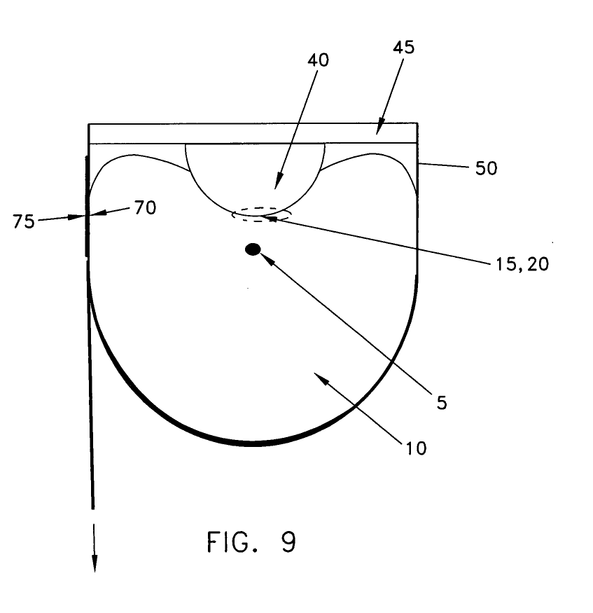

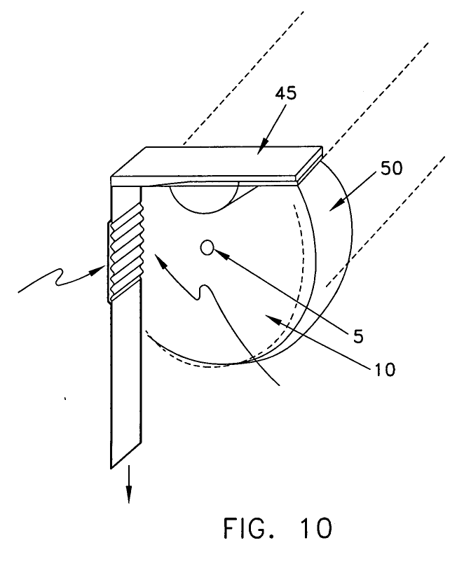

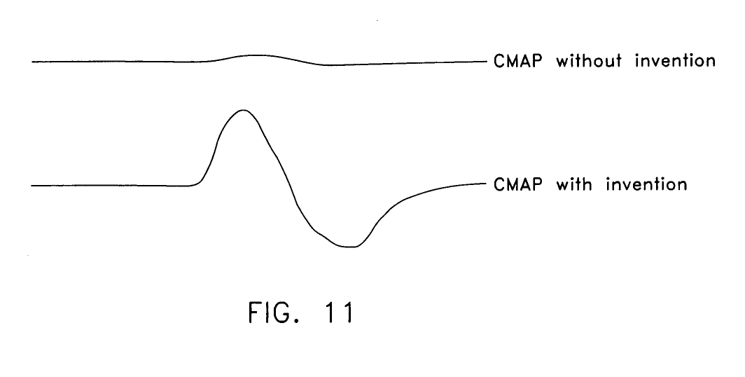

FIGS. 6-10 illustrate a preferred form of the present invention in use, bringing the stimulation electrodes closer to the nerve located within a limb, whereby to increase the current density of a typical nerve conduction test. More particularly, girding strap 35 comprises a U-shaped length 50 of non-stretching strap material made of a commonly available woven nylon or other suitable material. U-shaped length 50 is attached to rigid portion 45 at its ends 55, 60. U-shaped length 50 also comprises a pull segment 65. A section of elastic strap, 70, is attached to U-shaped length 50 intermediate its length so as to create a bulge 75 when elastic strap 70 is in its normal contracted condition (FIGS. 6 and 8). However, when pull segment 65 is pulled against the force of elastic strap 70, so that elastic strap 70 is stretched into an elongated condition (FIGS. 7, 9 and 10), shaped protrusion 40 can be driven into closer proximity to nerve 5. The bulge 75 acts as a stop to limit the degree to which shaped protrusion 40 can be driven into the tissue. The bulge 75 flattens under tension when the elastic strap is in its stretched condition (FIGS. 9 and 10). By using an elastic strap 70 which has a known spring constant, and by properly sizing bulge 75, the apparatus provides the medical practitioner with an appropriate indication that the proper degree of compression has been applied to the patient. This compression provides a force normal to the axis of the limb and, along with the area of the shaped protrusion, applies an appropriate pressure on the limb by the protrusion. Preferably the apparatus is constructed so that a compressive force in the range of approximately 2 to 6 pounds per square inch (PSI) is applied to the intervening tissue so as to cause the stimulus electrodes to be located closer to the nerve.

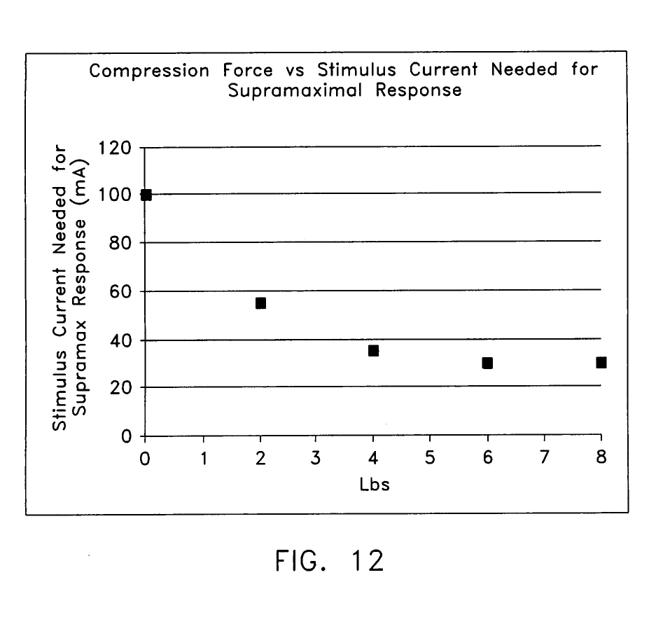

FIG. 11 illustrates the amplitude (in millivolts) of a typical Compound Muscle Action Potential (CMAP) with and without use of the present invention, for a given stimulation level. Significantly, as can be seen in FIG. 11, both the amplitude and waveform of the CMAP response are improved using the present invention.

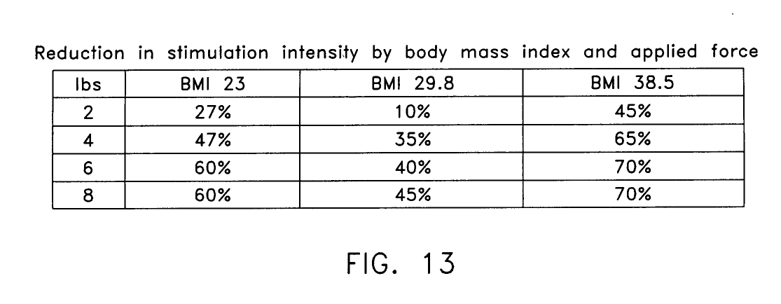

FIG. 12 shows the improvement obtained in CMAP amplitude with use of the present invention, plotting stimulus current against compression force in order to obtain a supramaximal CMAP response. In other words, FIG. 12 shows the reduction in stimulation intensity needed in order to maximally stimulate a nerve as pressure is applied over the stimulation site. The amount of current needed to stimulate the nerve is decreased as pressure is applied.

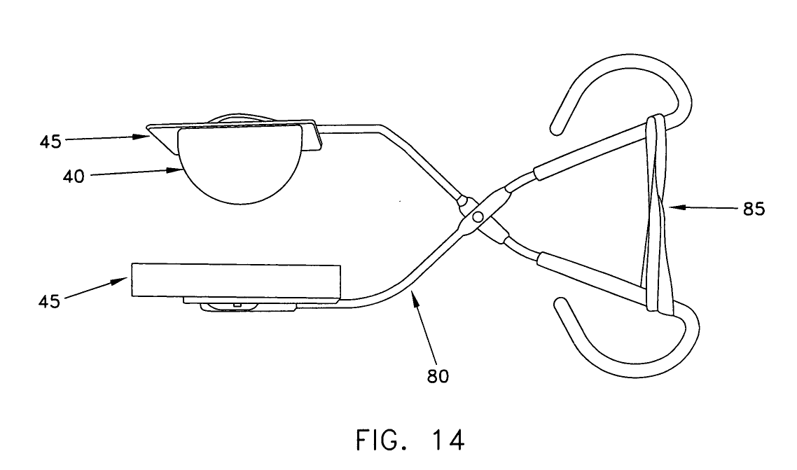

FIG. 13 shows the reduction in stimulation intensity, by body mass index and applied force. From FIG. 13 it will be seen that an increase in the CMAP response signal occurs for a patient of any Body Mass Index (BMI) when compression is applied, but the CMAP response signal has the greatest increase for a BMI that is greater than 38.5.

Looking next at FIG. 14, there is shown an alternate embodiment of the present invention. In this form of the invention, a compressive force is applied to shaped protrusion 40 via a spring-loaded clamp 80. In one preferred form of the invention, the spring-loaded clamp 80 has a pair of jaws each carrying a rigid portion 45, with one of the rigid portions carrying a shaped protrusion 40. The spring-loading of the clamp may be applied in a variety of ways, e.g., it may comprise a resilient member 85 such as an elastic strap or a tension spring. In this embodiment of the invention, the normal force is governed by the spring constant of the spring-loaded jaws of the clamp. This force is also normal to the axis of the limb and, along with the area of the protrusion, creates a pressure on the limb by the protrusion in a range of approximately 2 to 6 PSI that displaces the intervening tissue and that then causes the stimulus electrodes to be located closer to the nerve.

The configuration of shaped protrusion 40 depends on the area of the electrodes to be pushed into the flesh. A hemisphere, half cylinder and/or other shapes may be selected, depending on the requirements of the size and construction of the stimulation and detection means. The protrusion may be oriented in a direction relative to the electrodes so that it is non-directional as in the case of a hemispherical protrusion. A directional compression may be desirable if the stimulus electrodes are elongated in shape, as can be achieved in the case of a half cylinder.

It should also be appreciated that, if desired, the apparatus can be used to move the detection electrodes closer to the nerve, so as to improve response detection.

Thus, in one aspect of the invention, there is provided a compression device for optimally stimulating a nerve to evoke a bioelectrical signal, wherein the compression device comprises a spring element to create a compressive force, a shaped protrusion to push aside tissue, and a force indication element to facilitate tightening within a desired range, whereby the predetermined relationship between the spring element, shaped protrusion, and resultant force has been determined by electrophysiological evoked responses in a plurality of individuals so as to augment nerve stimulation and response detection.

The disclosed invention provides a novel approach for improving the efficiency of nerve stimulation in patients for a better assessment of neuromuscular function, without requiring highly trained personnel monitoring neuromuscular physiology.

### MODIFICATIONS

While the present invention has been described in terms of certain exemplary preferred embodiments, it will be readily understood and appreciated by one of ordinary skill in the art that it is not so limited, and that many additions, deletions and modifications to the preferred embodiments may be made within the scope of the invention as hereinafter claimed. Accordingly, the scope of the invention is limited only by the scope of the appended claims.

What is claimed is:

1. Apparatus for use in nerve conduction testing where a stimulating electrode is used to electrically stimulate a nerve and a detection electrode is used to detect an evoked neurological response, the apparatus comprising:
a shaped protrusion for positioning against an electrode; and
a compression mechanism for applying a force to the shaped protrusion so as to cause the shaped protrusion to press the electrode against the underlying tissue and compress the same, so as to move the electrode closer to the nerve and thereby augment nerve stimulation and response detection.

2. Apparatus according to claim 1 wherein the electrode comprises a stimulating electrode.

3. Apparatus according to claim 1 wherein the electrode comprises a detection electrode.

4. Apparatus according to claim 1 wherein the shaped protrusion comprises a distal surface having a cross-sectional profile selected from the group consisting of a hemisphere, a ball, a half cylinder, a full cylinder, a trapezoid, a rectangle, a polygon, and a post.

5. Apparatus according to claim 1 wherein the shaped protrusion comprises a proximal surface having a cross-sectional profile which is planar.

6. Apparatus according to claim 1 wherein a rigid portion is disposed between the shaped protrusion and the compression mechanism.

7. Apparatus according to claim 1 wherein the compression mechanism comprises an indication mechanism for indicating when the compression mechanism is applying an appropriate force on the shaped protrusion.

8. Apparatus according to claim 1 wherein the compression mechanism comprises a girding strap.

9. Apparatus according to claim 8 wherein the girding strap comprises a length of elastomeric material disposed intermediate the girding strap so as to (i) draw a portion of the girding strap into a loop when no cinching force is applied to the girding strap, and (ii) release the loop when an appropriate cinching force is applied to the girding strap, whereby to provide an indication mechanism for indicating when an appropriate cinching force is being applied to the girding strap.

10. Apparatus according to claim 1 wherein the compression mechanism comprises a clamp.

11. Apparatus according to claim 10 wherein the clamp comprises a first element for engaging the shaped protrusion and a second element for engaging the patient.

12. Apparatus according to claim 11 wherein the clamp further comprises a biasing mechanism for biasing the first element and the second element towards one another.

13. Apparatus according to claim 10 wherein the clamp comprises forceps, wherein one arm of the forceps engages the shaped protrusion, the other arm of the forceps engages the patient, and further wherein a tension mechanism is provided to urge the first arm towards the second arm.

14. A system for testing a nerve, comprising:
a stimulating electrode for electrically stimulating a nerve;
a detection electrode for detecting an evoked neurological response;
a shaped protrusion for positioning against an electrode; and
a compression mechanism for applying a force to the shaped protrusion so as to cause the shaped protrusion to press the electrode against the underlying tissue and compress the same, so as to move the electrode closer to the nerve and thereby augment nerve stimulation and response detection.

15. A method for augmenting nerve conduction testing where a stimulating electrode is used to electrically stimulate a nerve and a detection electrode is used to detect an evoked neurological response, the method comprising:
positioning a shaped protrusion against an electrode; and
positioning a compression mechanism against the shaped protrusion; and
activating the compression mechanism so as to apply a compressive force to the shaped protrusion, whereby to cause the shaped protrusion to press the electrode against the underlying tissue and compress the same, thereby moving the electrode closer to the nerve and augmenting nerve stimulation and response detection.
